(19) United States
(12) Patent Application Publication
(10) Pub. No.: US 2024/0342204 A1
(43) Pub. Date: Oct. 17, 2024

(54) METHOD OF TREATING VETERINARY VIRAL DISEASES

(71) Applicant: Intervet Inc., Rahway, NJ (US)

(72) Inventors: Edward Murray, Limerick, PA (US); Ralf Warrass, Alzey (DE); Joachim Ullrich, Stadecken-Elsheim (DE); Basav Hangalapura Nagaraj, Wageningen (NL); Willem Huisman, Nijmegen (NL)

(73) Assignee: Intervet Inc., Rahway, NJ (US)

(21) Appl. No.: 18/294,592

(22) PCT Filed: Aug. 5, 2022
(86) PCT No.: PCT/EP2022/072074

§ 371 Date: Feb. 2, 2024

(30) Foreign Application Priority Data
Aug. 6, 2021 (EP) 21190172.3
Jul. 19, 2022 (EP) 22185716.2

(51) Int. Cl. A61K 31/7068; A61P 31/14; A61P 31/16; A61P 37/04

(52) U.S. Cl. CPC A61K 31/7068; A61P 31/14; A61P 31/16; A61P 37/04

(57) ABSTRACT

A method of preventing or treating a viral disease in an animal comprising administering to the animal a pharmaceutical composition comprising an antiviral nucleoside β-D-N(4)-hydroxycytidine (NHC) or a prodrug or salt thereof and a pharmaceutically acceptable carrier and wherein the viral disease is Porcine Reproductive and Respiratory Syndrome (PRRS), Bovine Respiratory Disease (BRD), Equine Influenza, Canine Distemper, Canine Respiratory Disease, Feline Infectious Peritonitis, Feline Viral Rhinotracheitis, or Feline Immunodeficiency.

### FIELD OF THE INVENTION

The present invention relates to antiviral nucleoside compounds and pharmaceutical compositions for use in the treatment or prevention of viral infections and viral diseases in animals.

### BACKGROUND

The development of antiviral drugs for use in veterinary medicine has been constrained by the need to identify specific viral targets with increased selectivity and reduced side effects. As a result, very few antiviral drugs have been successfully developed for veterinary use. See Dal Pozzo & Thiry, Rev. sci. tech. Off int. Epiz., 2014, 33 (3), 791-801.

Porcine Reproductive and Respiratory Syndrome (PRRS) is a viral disease characterized by two overlapping clinical presentations, reproductive impairment or failure in breeding animals, and respiratory disease in pigs of any age. PRRS is the most economically significant disease to affect US swine production. There is no specific treatment for PRRS. Broad-spectrum antibiotics may be useful in controlling secondary infections. Anti-inflammatory products (e.g. aspirin) are commonly administered during acute disease. Other helpful techniques include early weaning and isolation of piglets, various PRRS vaccination protocols, regular serologic monitoring, testing (ELISA, PCR and IFA) and removal of persistent carriers in herds with <10% infection and improving biosecurity. (See Swine Manual, Iowa State University, College of Veterinary Medicine, Swine Manual, https://vetmed.iastate.edu/vdpam/FSVD/swine/index-diseases/porcine-reproductive, accessed Apr. 22, 2021).

Bovine Respiratory Disease (BRD) also known as “shipping fever”, is the most common and costly disease affecting the North American beef cattle industry. In the broadest sense, BRD refers to any disease of the upper or lower respiratory tracts. BRD in cattle is commonly associated with infections of the lungs causing pneumonia in calves that have recently been weaned or recently arrived at the feedlot (which is why it is often referred to as shipping fever). BRD or shipping fever, is most prevalent within the first weeks of arrival to the feedlot, but it can occur later in the feeding period and is also seen in calves on pasture. Infectious agents or pathogens that are necessary for causing the disease can broadly be categorized as viruses, bacteria and parasites. Viruses known to cause BRD are bovine herpes virus (IBR); bovine parainfluenza virus (PI-3); bovine respiratory syncytial virus (BRSV); bovine viral diarrhea virus (BVD), and bovine coronavirus (BCV). (See “Bovine Respiratory Disease” Beef Cattle Research Council, http://www.beefresearch.ca/research-topic.cfm/bovine-respiratory-disease-38, revised Oct. 2, 2019, accessed Apr. 22, 2021). US 2017/0260147 A1 discloses guanidine derivative compounds which demonstrate activity against bovine coronavirus (see Examples 43 and 44). β-D-N(4)-hydroxycytidine (NHC) inhibited the production of cytopathic bovine viral diarrhea virus (BVDV) RNA in a dose-dependent manner with a 90% effective concentration (EC₉₀) of 5.4 μM, an observation that was confirmed by virus yield assays (EC₉₀=2 μM) (See Stuyver et al, ANTIMICROBIAL AGENTS AND CHEMOTHERAPY, Vol. 47, No. 1, January 2003, p. 244-254).

Equine influenza is a common, highly contagious respiratory disease of equids with a near-global distribution. The most common clinical signs of EIV infection in equids are fever, lethargy, anorexia, nasal discharge, and a nonproductive dry cough. Mortality rates are generally low during EIV outbreaks; death is most common among foals or equids with preexisting poor health. The risk of EIV infection is not limited to equids; dogs, cats and humans are also susceptible. This disease has been found difficult to control through vaccination because vaccination does not prevent viral shedding. (See Sack et al. EID Journal, Vol. 25 (6) June 2019).

Canine distemper is a highly contagious viral disease caused by a paramyxovirus. It is seen in dogs around the world, but it can also affect ferrets, racoons, skunks, grey foxes, and many other animals. Canine distemper affects the gastrointestinal, respiratory, skin, immune, and central nervous systems. There is no cure for canine distemper. Vaccination is recommended to prevent canine distemper in adult dogs and puppies. (See A. Flowers “Canine Distemper”, https://pets.webmd.com/dogs/canine-distemper #1, reviewed Feb. 10, 2021, accessed Apr. 23, 2021).

Canine infectious respiratory disease complex (CIRDC) refers to a syndrome of diseases that can be caused by several different bacterial and viral pathogens. Historically, the most common pathogens associated with CIRDC have been canine parainfluenza virus (CPIV), canine adenovirus type 2 (CAV-2), and Bordetella bronchiseptica. Outbreaks of novel pathogens, including canine herpesvirus-1 (CHV-1) and canine influenza virus (CIV) have been reported. Treatment of dogs with signs of CIRDC typically involves supportive care. Vaccines are available for many common CIRDC pathogens and are recommended for dogs that have a risk of exposure. However, the available vaccines do not convey sterilizing immunity and there are no specific therapies available for viral CIRDC pathogens. (See K. Reagan & J. Sykes, Vet Clin Small Anim. 50 (2020) 405-418).

Feline infectious peritonitis (FIP) is a viral disease caused by a feline coronavirus that affects wild and domestic cats. Feline coronavirus is very common and usually doesn't cause any serious issues, aside from mild diarrhea. But when the feline coronavirus changes to a specific strain of the coronavirus, FIP can develop. In about 10% of infected cats, the virus will multiply and mutate, resulting in an infection known as feline infectious peritonitis virus (FIPV) that spreads throughout the cat's body. It can cause an extreme inflammatory reaction in the tissues surrounding the abdomen, kidney, or brain. Although FIP is not believed to be contagious, it is a very serious disease. When a cat gets FIP, it is progressive and almost always fatal. FP has long been considered an untreatable disease. It wasn't until recently that antiviral drugs were introduced to help treat FIP. These drugs are not yet approved by the Food and Drug Administration (FDA), and their long-term effectiveness is still unknown. While a FIP vaccine is available, it has not been proven effective and is not recommended by the American Association of Feline Practitioners Feline Vaccine Advisory Panel. (see https://pets.webmd.com/cats/cat-fip-feline-infectious-peritonitis #1, reviewed Feb. 12, 2021, accessed Apr. 23, 2021). The nucleoside analog GS-441524 has been found to strongly inhibit feline infectious peritonitis (FIP) virus in tissue culture and experimental cat infection studies. (See Murphy et al., Veterinary Microbiology 219 (2018) 226-233).

Feline viral rhinotracheitis (FVR) is a common, worldwide respiratory disease of cats caused by felid herpesvirus 1 (FeHV-1). The disease causes an impairment of pulmonary defense mechanisms predisposing cats to secondary bacterial pneumonia or to a coinfection with feline calicivirus. The virus also can remain latent in ganglia. The vast majority of cats that recover from FVR become carriers and shed FeHV-1, either spontaneously or following stress. Susceptible animals, particularly kittens with low maternal immunity, become infected after exposure to a diseased or carrier cat. Replication of FeHV-1 in the nasal, conjunctival, pharyngeal, and, to a lesser extent, tracheal epithelium causes degeneration and exfoliation of cells. (See Alfonso López, Shannon A. Martinson, Pathologic Basis of Veterinary Disease, Elsevier Inc. Sixth Edition, James F. Zachary, Editor, Chapter 9, pp 471-560, 2017).

WO 2012/152317 discloses the use of derivatives of purine and pyrimidine in the treatment of feline viral diseases. Feline immunodeficiency virus (FIV) and feline leukemia (FeLV) infections are common among domestic cats (Felis cat us) with greater than 8% infected with FIV and 14% infected with FeLV infected (see Table 1 of WO 2012/152317). From 2002 until 2017, a FIV vaccination was available in the United States and Canada. However, this vaccine has since been discontinued because it offered limited protection, raised the risk of vaccine site sarcoma and led to false positives in the FIV antibody test (See N. Stilwell “What is FIV and Why is the FIV Vaccine No Longer Available” https://www.petmd.com/cat/care/what-fiv-and-why-fiv-vaccine-no-longer-available, published Jun. 21, 2019, updated Jul. 9, 2020, accessed Apr. 22, 2021).

US 2015/0018427 A1 demonstrates the use of a compound of the amantadine family for the treatment or prevention of a parvovirus infection in a human or animal.

β-D-N(4)-hydroxycytidine (NHC, 1-((2R,3R,4S,5R)-3,4-dihydroxy-5-(hydroxymethyl)tetrahydrofuran-2-yl)-4-(hydroxyamino)pyrimidin-2(1H)-one) was found to have antipestivirus and antihepacivirus activities. ANTIMICROB AGENTS CHEMOTHER, 2003, 47(1):244-54. β-D-N(4)-hydroxycytidine,

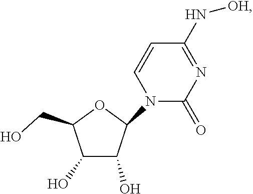 [SMILES: O=c1nc(NO)ccn1[C@@H]1O[C@H](CO)[C@@H](O)[C@H]1O]

derivatives, and methods for making the same are illustrated in PCT International Patent Application No. PCT/US2015/066144, which published as PCT International Patent Application Publication No. WO2016/106050, and U.S. patent application Ser. No. 15/537,087, which published as United States Patent Application Publication No. US2019/0022116, and U.S. patent application Ser. No. 16/921,359, which published as United States Patent Application Publication No. US 2021-0060050 A1. NHC is known to tautomerize, and it is also known as (Z)-1-((2R,3R,4S,5R)-3,4-dihydroxy-5-(hydroxymethyl)tetrahydrofuran-2-yl)-4-(hydroxyimino)-3,4-dihydropyrimidin-2(1H)-one:

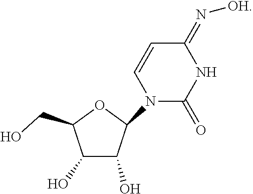 [SMILES: O=c1[nH]/c(=N\O)ccn1[C@@H]1O[C@H](CO)[C@@H](O)[C@H]1O]

WO 2016/106050 A1 discloses N4-hydroxycytidine derivatives, compositions, and their use in the treatment and prophylaxis of viral infections.

WO 2019/113462 A1 suggests certain N4-hydroxycytidine derivatives and pharmaceutical compositions for use in the treatment or prophylaxis of viral infections, such as Eastern, Western, and Venezuelan Equine Encephalitis (EEE, WEE and VEE, respectively), Chikungunya fever (CHIK), Ebola, Influenza, RSV, and Zika virus infection.

Methods for preparing β-D-N(4)-hydroxycytidine (NHC), and derivatives thereof are disclosed in PCT International Patent Application No. PCT/US2019/021168, which published as PCT International Patent Application Publication No. WO2019/173602, and in U.S. patent application Ser. No. 16/755,779, which published as United States Patent Application Publication No. US 2020-0276219 A1.

There are limited or no antiviral therapies available for treating or preventing Porcine Reproductive and Respiratory Syndrome (PRRS), Bovine Respiratory Disease (BRD), Equine Influenza, Canine Distemper, Canine Respiratory disease, Feline Infectious Peritonitis, Feline Viral Rhinotracheitis, or Feline Immunodeficiency in animals. There is a need among livestock producers and pet owners for such therapies. None of the cited references disclose the use of N4-hydroxycytidine or its prodrugs or salts in the treatment or prevention of these diseases.

Adenosine nucleoside analogue GS-441524 has been used as an antiviral treatment of cats with clinically diagnosed neurological feline infectious peritonitis (see Dickinson et al., J Vet Intern Med. 2020 July; 34(4):1587-1593. doi: 10.11111/jvim.15780. Epub 2020 May 22, PMID: 32441826)

Efficacy and safety of the nucleoside analog GS-441524 for treatment of cats with naturally occurring feline infectious peritonitis has been investigated. (See Pedersen et al., J Feline Med Surg. 2019 April; 21(4):271-281. doi: 10.1177/1098612X19825701. Epub 2019 Feb. 13. PMID: 30755068; PMCID PMC6435921.

The nucleoside analog GS-441524 has been found to strongly inhibit feline infectious peritonitis (FIP) virus in tissue culture and experimental cat infection studies. See Murphy et al., Vet Microbiol. 2018 June; 219:226-233. doi: 10.1016/j.vetmic.2018.04.026. Epub 2018 Apr. 22. PMID: 29778200; PMCID PMC7117434.

Treating Cats with Feline Infectious Peritonitis with an Oral Multi-Component Drug Containing GS-441524 has been demonstrated. Krentz D, Zenger K, Alberer M, Felten S, Bergmann M, Dorsch R, Matiasek K, Kolberg L, Hofmann-Lehmann R, Meli M L, Spiri A M, Horak J, Weber S, Holicki C M, Groschup M H, Zablotski Y, Lescrinier E, Koletzko B, von Both U, Hartmann K. Curing Cats with Feline Infectious Peritonitis with an Oral Multi-Component Drug Containing GS-441524. Viruses. 2021 Nov. 5; 13(11):2228. doi: 10.3390/v13112228. PMID: 34835034; PMCID: PMC8621566.

### SUMMARY OF THE INVENTION

A method of preventing or treating a viral disease in an animal comprising administering to the animal a pharmaceutical composition comprising an antiviral nucleoside β-D-N(4)-hydroxycytidine (NHC) or a prodrug or salt thereof and a pharmaceutically acceptable carrier and wherein the viral disease is Porcine Reproductive and Respiratory Syndrome (PRRS), Bovine Respiratory Disease (BRD), Equine Influenza, Canine Distemper, Canine Respiratory Disease, Feline Infectious Peritonitis, Feline Viral Rhinotracheitis, or Feline Immunodeficiency.

### DESCRIPTION OF THE FIGURES

FIG. 1—Antiviral dose response curve of NHC against FIPV infected feline kidney cells. NHC is protective against viral induced CytoPathic Effect (CPE) in a dose dependent manner.

FIG. 2 shows NHC activity against Feline Calicivirus.

FIG. 3 shows antiviral activity of NHC against Swine rotavirus strains G5P7 and G9P7.

FIG. 4 shows antiviral activity of NHC against Swine influenza A viruses H3N2 and H1N1pdm.

### DETAILED DESCRIPTION OF THE INVENTION

Viral infections in animals have a large economic impact to the animal health industry. These infections are traditionally managed and prevented successfully by vaccination, testing and culling. Currently viral infections in animal are not targeted by antiviral small molecule drugs.

There are several challenges for the use of antiviral drugs in animal health. The treatment or prevention of viral infections in animals are cost sensitive. This is especially true in livestock animals (such as cattle, swine, poultry and aquaculture). In many jurisdictions, culling of the infected animals is a legal requirement for some viral diseases (e.g. foot-and-mouth-disease). Convenience is another challenge for animal health antiviral drugs. For example, in many species a one-shot dose might be a prerequisite. Furthermore, treatment with antiviral drugs must not interfere with vaccination strategy for the animal. For food producing animals, the antiviral drug must be able to be administered to establish a minimal residual level (MRL) in edible tissue. Finally, the antiviral drug must have a clean toxicity profile for the target animal and be safe for the humans who administer the drug.

Yet another challenge is the efficacy level required. Must the antiviral drug be capable of virus elimination or cure? Is it required to simply maintain the viral load or is it necessary to reduce the viral load in order to treat or prevent the viral disease? Should viral shedding be reduced or eliminated to prevent transmission of the disease from one animal to another?

The synthetic nucleoside derivative N4-hydroxycytidine and prodrugs and salts thereof are believed to exerts their antiviral action through introduction of copying errors during viral RNA replication. As noted above, activity has also been demonstrated against coronaviruses including SARS, MERS and SARS-CoV-2. The presumed mechanism of action (MOA) is by “error catastrophe” in which the error rate of replication is greater than the allowed error threshold to sustain the viral population.

It is contemplated that the synthetic nucleoside derivative N4-hydroxycytidine and prodrugs and salts thereof are effective antiviral drugs to treat or prevent viral infections that cause viral disease, wherein the viral disease is Porcine Reproductive and Respiratory Syndrome (PRRS), Bovine Respiratory Disease (BRD), Equine Influenza, Canine Distemper, Canine Respiratory Disease, Feline Infectious Peritonitis, Feline Viral Rhinotracheitis, or Feline Immunodeficiency.

It is also contemplated that the synthetic nucleoside derivative N4-hydroxycytidine and prodrugs and salts thereof are effective antiviral drugs to treat or prevent viral infections that cause viral disease, wherein the viral disease is Porcine Reproductive and Respiratory Syndrome (PRRS), Bovine Respiratory Disease (BRD), Canine Distemper, Canine Respiratory Disease, Feline Infectious Peritonitis, Feline Viral Rhinotracheitis, or Feline Immunodeficiency.

Moreover, it is contemplated that the synthetic nucleoside derivative N4-hydroxycytidine and prodrugs and salts thereof are effective antiviral drugs to stabilize or reduce the viral load of a viral infection in an animal wherein the viral infection is from Porcine Reproductive and Respiratory Syndrome virus (PRRS-1/PRRS-2), Bovine Leukemia virus (BLV), Bovine Corona Virus (BCV), Bovine Respiratory Syncytial Virus (BRSV), Bovine Rotavirus, Bovine Parainfluenza Virus 3, Bovine Viral Diarrhea Virus (BVDV), Equine Influenza Virus, Canine Distemper virus, Canine Influenza virus (CIV), Coronaviridae (FIPV), Feline Calicivirus, or Feline Immunodeficiency virus (FIV).

The methods or uses described herein also address one or more of the challenges of animal health antiviral therapies discussed above. Specifically, the synthetic nucleoside derivative N4-hydroxycytidine and prodrugs and salts thereof when administered to animals maintain or reduce the viral load in the animal and reduce or eliminate viral shedding from the animal.

This results in a suppressed virus spread and blocks transmission to untreated contact animals.

### Definitions

Certain technical and scientific terms are specifically defined below. Unless specifically defined elsewhere in this document, all other technical and scientific terms used herein have the meaning commonly understood by one of ordinary skill in the art to which this disclosure relates.

As used herein, including the appended claims, the singular forms of words such as “a,” “an,” and “the,” include their corresponding plural references unless the context clearly dictates otherwise.

The terms “administration of” and or “administering” a compound should be understood to include providing a compound described herein, or a pharmaceutically acceptable salt thereof, and compositions of the foregoing to a subject.

As used herein, the terms “at least one” item or “one or more” item each include a single item selected from the list as well as mixtures of two or more items selected from the list.

The term “pharmaceutically acceptable carrier” refers to any inactive substance that is suitable for use in a formulation for the delivery of a therapeutic agent. A carrier may be an antiadherent, binder, coating, disintegrant, filler or diluent, lubricant, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like), dextrose, vegetable oils (such as olive oil), saline, buffer, buffered saline, and isotonic agents such as sugars, polyalcohols, sorbitol, and sodium chloride.

The animal may be one or more selected from the group consisting of bovine (e.g., cows), porcine (e.g., pigs), ovine (e.g., sheep), capra (e.g., goats), equine (e.g., horses), canine (e.g., domestic dogs), feline (e.g., house cats), Lagomorpha (rabbits), rodents (e.g., rats or mice), Procyon lotor (e.g., raccoons).

As used herein, the terms “treatment” and “treating” refer to all processes in which there may be a slowing, interrupting, arresting, controlling, or stopping of the progression of a disease or disorder described herein. The terms do not necessarily indicate a total elimination of all disease or disorder symptoms.

As used herein, the terms “prophylaxis” and “antiviral prophylaxis” refer to all processes intended to prevent disease. Prophylaxis may occur prior to exposure to a viral infection or after a potential exposure to a viral infection.

The therapeutic agents and compositions used in the present methods can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route.

The administration may be by oral, topical, or injectable route.

The term “simultaneous administration” as used herein in relation to the administration of medicaments refers to the administration of medicaments such that the individual medicaments are present within a subject at the same time. In addition to the concomitant administration of medicaments (via the same or alternative routes), simultaneous administration may include the administration of the medicaments (via the same or an alternative route) at different times.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

It is understood that wherever embodiments are described herein with the language “comprising,” otherwise analogous embodiments described in terms of “consisting of” and/or “consisting essentially of” are also provided.

Unless expressly stated to the contrary, all ranges cited herein are inclusive, i.e., the range includes the values for the upper and lower limits of the range as well as all values in between. All ranges also are intended to include all included sub-ranges, although not necessarily explicitly set forth. As an example, temperature ranges, percentages, ranges of equivalents, and the like described herein include the upper and lower limits of the range and any value in the continuum there between.

Numerical values provided herein, and the use of the term “about”, may include variations of ±1%, ±2%, 3%, 4%, 5%, and ±10% and their numerical equivalents. “About” when used to modify a numerically defined parameter (e.g., the dose of an antiviral nucleoside, or the length of treatment time with a combination therapy described herein) means that the parameter may vary by as much as 10% below or above the stated numerical value for that parameter; where appropriate, the stated parameter may be rounded to the nearest whole number. For example, a dose of about 5 mg/kg may vary between 4.5 mg/kg and 5.5 mg/kg. In addition, the term “or,” as used herein, denotes alternatives that may, where appropriate, be combined; that is, the term “or” includes each listed alternative separately as well as their combination.

As used herein, “antiviral nucleosides” means any nucleoside chemical compound that exhibits antiviral activity.

Viral load as used herein means a measurement of the amount of a virus in an organism, typically in the bloodstream, usually stated in virus particles per milliliter.

Viral shedding refers to the expulsion and release of virus progeny following successful reproduction during a host-cell infection.

Sterilizing immunity means that the immune system is able to stop a pathogen, including viruses, from replicating within the animal.

Prodrug as used here in means a biologically inactive compound which can be metabolized in the body to produce a drug. The prodrugs of the present invention can have any form suitable to the formulator, for example, esters, more specifically alkylesters, are non-limiting common prodrug forms. Examples of prodrugs of the antiviral nucleoside NHC are N-hydroxycytidine 5′-(2-methylpropanoate)(Compound A):

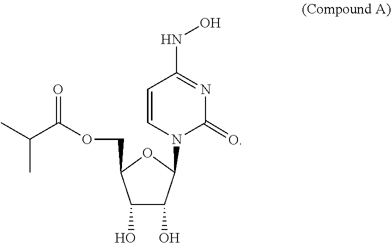 [SMILES: CC(C)C(=O)OC[C@H]1O[C@@H](n2ccc(NO)nc2=O)[C@H](O)[C@@H]1O]

and uridine 4-oxime 5′-(2-methylpropanoate) (Compound B), also known as {(2R,3S,4R,5R)-3,4-dihydroxy-5-[4-(hydroxyimino)-2-oxo-3,4-dihydropyrimidin-1(2H)-yl]oxolan-2-yl}methyl 2-methylpropanoate):

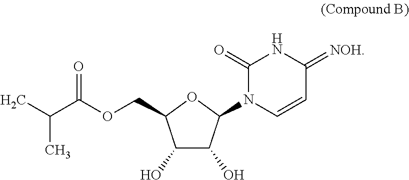

### Salts

The compounds of the present invention can be employed in the form of pharmaceutically acceptable salts. Those skilled in the art will recognize those instances in which the compounds of the invention may form salts. Examples of such compounds are described herein by reference to possible salts. Such reference is for illustration only.

Pharmaceutically acceptable salts can be used with compounds for treating patients. Nonpharmaceutical salts may, however, be useful in the preparation of intermediate compounds.

The term “pharmaceutically acceptable salt” refers to a salt (including an inner salt such as a zwitterion) that possesses effectiveness similar to the parent compound and that is not biologically or otherwise undesirable (e.g., is neither toxic nor otherwise deleterious to the recipient thereof). Thus, an embodiment of the invention provides for use of pharmaceutically acceptable salts of the compounds. The term “salt(s)”, as employed herein, denotes any of the following: acidic salts formed with inorganic and/or organic acids, as well as basic salts formed with inorganic and/or organic bases. Salts of compounds of the invention may be formed by methods known to those of ordinary skill in the art, for example, by reacting a compound of the invention with an amount of acid or base, such as an equivalent amount, in a medium such as one in which the salt precipitates or in aqueous medium followed by lyophilization.

Acids that are generally considered suitable for the formation of pharmaceutically useful salts from basic pharmaceutical compounds are discussed, for example, by P. Stahl et al, Cam ille G. (eds.), Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

The present disclosure relates to methods of treating a viral disease as defined herein, wherein the method comprises administering to an animal in need thereof a therapy that comprises the antiviral nucleoside NHC or a prodrug or a salt thereof. The present disclosure relates to methods of treating a viral disease, wherein the method comprises administering to a subject in need thereof a therapy that comprises an antiviral nucleoside NHC or a prodrug or a salt thereof; wherein the viral disease is selected from the group consisting of Porcine Reproductive and Respiratory Syndrome (PRRS), Bovine Respiratory Disease (BRD), Equine Influenza, Canine Distemper, Canine Respiratory Disease, Feline Infectious Peritonitis, Feline Viral Rhinotracheitis, or Feline Immunodeficiency.

Porcine rotavirus B as primary causative agent of diarrhea outbreaks in newborn piglets. Rotavirus (RV) is considered a major cause of acute viral gastroenteritis in young animals. Miyabe et al., Scientific Reports, (2020) 10:22002, https://doi.org/10.1038/s41598-020-78797-y

Porcine or swine influenza is a respiratory disease of pigs caused by type A influenza viruses that regularly cause outbreaks of influenza in pigs. The main swine influenza viruses circulating in U.S. pigs in recent years have been, swine triple reassortant (tr) H1N1 influenza virus, trH3N2 virus, and trH1N2 virus. Centers for Disease Control and Prevention, National Center for Immunization and Respiratory Diseases (NCIRD) https://www.cdc.gov/flu/swineflu/keyfacts_pigs.htm (Page last reviewed: Aug. 19, 2014).

Treatment of such a viral disease results in a reduction of clinical signs in the treated animal and reduction of mortality in animals showing signs of severe disease. In another embodiment the treatment accelerates recovery from clinical symptoms.

The present disclosure relates to methods of providing antiviral prophylaxis, wherein the method comprises administering to a subject in need thereof a composition that comprises an antiviral nucleoside NHC or a prodrug or a salt thereof; wherein the viral disease is selected from the group consisting of Porcine Reproductive and Respiratory Syndrome (PRRS), Bovine Respiratory Disease (BRD), Equine Influenza, Canine Distemper, Canine Respiratory Disease, Feline Infectious Peritonitis, Feline Viral Rhinotracheitis, or Feline Immunodeficiency. Antiviral prophylaxis is especially useful when the antiviral nucleoside NHC or a prodrug or a salt thereof is administered at the peak of virus replication but before onset of virus shedding.

Additionally, the present disclosure relates to methods of treating a viral infection, wherein the method comprises administering to a subject in need thereof a therapy that comprises an antiviral nucleoside NHC or a prodrug or a salt thereof; wherein the viral infection is selected from the group consisting of Porcine Reproductive and Respiratory Syndrome virus (PRRS-1/PRRS-2), Bovine Leukemia virus (BLV), Bovine Corona Virus (BCV), Bovine Respiratory Syncytial Virus (BRSV), Bovine Rotavirus, Bovine Parainfluenza Virus 3, Bovine Viral Diarrhea Virus (BVDV), Equine Influenza Virus, Canine Distemper virus, Canine Influenza virus (CIV), Coronaviridae (FIPV), Feline Calicivirus, or Feline Immunodeficiency virus (FIV).

Furthermore, present disclosure relates to methods of providing antiviral prophylaxis, wherein the method comprises administering to a subject in need thereof a composition that comprises an antiviral nucleoside NHC or a prodrug or a salt thereof; wherein the viral infection is selected from the group consisting of Porcine Reproductive and Respiratory Syndrome virus (PRRS-1/PRRS-2), Bovine Leukemia virus (BLV), Bovine Corona Virus (BCV), Bovine Respiratory Syncytial Virus (BRSV), Bovine Rotavirus, Bovine Parainfluenza Virus 3, Bovine Viral Diarrhea Virus (BVDV), Equine Influenza Virus, Canine Distemper virus, Canine Influenza virus (CIV), Coronaviridae (FIPV), Feline Calicivirus, or Feline Immunodeficiency virus (FIV).

Products provided as therapies and prophylaxis may include a composition comprising an antiviral nucleoside NHC or a prodrug or a salt thereof in a composition. The therapy may also comprise one or more additional therapeutic agents. The one or more additional active agents may be administered with antiviral nucleoside (co-administered) or administered separately from the antiviral nucleoside, in a different dosage form. That is, the additional active agent(s) may be administered in a single dosage form with the antiviral nucleoside, or the additional active agent(s) may be administered in separate dosage form(s) from the dosage form containing the antiviral nucleoside. The therapies disclosed herein may be used in combination with one or more other active agents, including but not limited to, antiviral agents that are used in the prevention, treatment, control, amelioration, or reduction of risk of a particular disease or condition (e.g., viral infection). In one embodiment, the antiviral nucleoside NHC or a prodrug or a salt thereof disclosed herein is combined with one or more other antiviral agents for use in the prevention, treatment, control amelioration, or reduction of risk of a particular disease or condition for which the antiviral nucleoside NHC or a prodrug or a salt thereof disclosed herein are useful. Such other active agents may be administered, by a route and in an amount commonly used therefor, contemporaneously or sequentially with a compound of the present disclosure. When the therapies disclosed herein are used contemporaneously with one or more other active agents, the antiviral nucleoside may be administered either simultaneously with, or before or after, one or more other active agent(s). The antiviral nucleoside may be administered separately, by the same or different route of administration, or together in the same pharmaceutical composition as the other agent(s).

The dosage amount of the antiviral nucleoside NHC or a prodrug or a salt thereof may be varied and will depend upon the therapeutically effective dose of each agent.

Generally, a therapeutically effective dose of each will be used. Combinations including at least one antiviral nucleoside, and other active agents will generally include a therapeutically effective dose of each active agent. In such combinations, the antiviral nucleosides disclosed herein and other active agents may be administered separately or in conjunction. In addition, the administration of one element may be prior to, concurrent with, or subsequent to the administration of other agent(s). In one embodiment, this disclosure provides an antiviral nucleoside, and at least one other active agent as a combined preparation for simultaneous, separate or sequential use in therapy. In one embodiment, the therapy is the treatment of a viral disease, wherein the viral disease is selected from the group consisting of Porcine Reproductive and Respiratory Syndrome (PRRS), Bovine Respiratory Disease (BRD), Equine Influenza, Canine Distemper, Canine Respiratory Disease, Feline Infectious Peritonitis, Feline Viral Rhinotracheitis, or Feline Immunodeficiency. In one embodiment, the therapy is antiviral prophylaxis, such as for potential infection, either pre-exposure or post-exposure to a viral infection, wherein the viral infection is selected from the group consisting of Porcine Reproductive and Respiratory Syndrome (PRRS), Bovine Respiratory Disease (BRD), Equine Influenza, Canine Distemper, Canine Respiratory Disease, Feline Infectious Peritonitis, Feline Viral Rhinotracheitis, or Feline Immunodeficiency. In another embodiment, the therapy is the treatment of a viral disease, wherein the virus is selected from the group consisting of Porcine Reproductive and Respiratory Syndrome virus (PRRS-1/PRRS-2), Bovine Leukemia virus (BLV), Bovine Corona Virus (BCV), Bovine Respiratory Syncytial Virus (BRSV), Bovine Rotavirus, Bovine Parainfluenza Virus 3, Bovine Viral Diarrhea Virus (BVDV), Equine Influenza Virus, Canine Distemper virus, Canine Influenza virus (CIV), Coronaviridae (FIPV), Feline Calicivirus, or Feline Immunodeficiency virus (FIV).

The additional active agent(s) may be one or more agents selected from the group consisting of antiviral compounds, antigens, adjuvants, anti-cancer agents, CTLA-4 agonists, LAG-3 agonists, PD-1 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, antibiotics, anti-metabolites, retinoids, steroids, and immunomodulatory agents, including but not limited to antiviral vaccines. It will be understood the descriptions of the above additional active agents, and of those listed below, may be overlapping. It will also be understood that the treatment combinations are subject to optimization, and it is understood that the best combination to use of the antiviral nucleoside, and one or more additional active agents will be determined based on the individual animal needs.

Vaccine therapies that may be used in combination with the therapies disclosed herein include but are not limited to inactivated vaccines, live-attenuated vaccines, recombinant vaccines, replication-deficient viral vector vaccines, mRNA-based vaccines, DNA vaccines, nanoparticle vaccines, non-replicating viral vectors, self-replicating RNA vaccines, self-amplifying RNA vaccines, protein subunit vaccines, Ii-Key peptide COVID-19 vaccines, gp96-based vaccines, intranasal vaccines, and mRNA lipid nanoparticle (mRNA10 LNP) vaccine.

The disclosure further relates to methods of treating a viral disease, said method comprising administering to a subject in need thereof a therapy that comprises an antiviral nucleoside NHC or a prodrug or a salt thereof; wherein the antiviral nucleoside is administered once daily for 1 to 10 days, such as for 1 day, 2 days, 3 days, 4 days, 5 days, 6 days, 7 days, 8 days, 9 days, or 10 days. In specific embodiments, the antiviral nucleoside NHC or a prodrug or a salt thereof is administered once daily for 3 to 5 days, such as for 3 days, 4 days, or 5 days.

The disclosure further relates to methods of treating a viral disease, said method comprising administering to a subject in need thereof a therapy that comprises an antiviral nucleoside NHC or a prodrug or a salt thereof; wherein the antiviral nucleoside is administered twice daily for 1 to 10 days, such as for 1 day, 2 days, 3 days, 4 days, 5 days, 6 days, 7 days, 8 days, 9 days, or 10 days. In specific embodiments, the antiviral nucleoside is administered twice daily for 3 to 5 days, such as for 3 days, 4 days, or 5 days.

In embodiments of the treatment methods disclosed herein, the antiviral nucleoside NHC or a prodrug or a salt thereof is administered once daily, as a single dose of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside NHC or a prodrug or a salt thereof is administered once daily, as a single dose of an amount of about 50 mg, about 100 mg, about 200 mg, about 300 mg, about 400 mg, about 500 mg, about 600 mg, about 700 mg, about 800 mg, about 1000 mg, about 1200 mg, about 1400 mg, or about 1600 mg. In aspects of such embodiments, the antiviral nucleoside NHC or a prodrug or a salt thereof is administered once daily, as a single dose of an amount of about 200 mg. In aspects of such embodiments, the antiviral nucleoside is administered once daily, as a single dose of an amount of about 300 mg. In aspects of such embodiments, the antiviral nucleoside is administered once daily, as a single dose of an amount of about 400 mg. In aspects of such embodiments, the antiviral nucleoside is administered once daily, as a single dose of an amount of about 500 mg. In aspects of such embodiments, the antiviral nucleoside is administered once daily, as a single dose of an amount of about 600 mg. In aspects of such embodiments, the antiviral nucleoside is administered once daily, as a single dose of an amount of about 700 mg. In aspects of such embodiments, the antiviral nucleoside NHC or a prodrug or a salt thereof is administered once daily, as a single dose of an amount of about 800 mg.

In embodiments of the treatment methods disclosed herein, the antiviral nucleoside NHC or a prodrug or a salt thereof is administered twice daily, as individual doses of an amount of from about 50 mg to about 1600 mg, such as from about 100 mg to about 1400 mg, from about 200 mg to about 1200 mg, from about 300 mg to about 1000 mg, or from about 400 mg to about 800 mg. In aspects of such embodiments, the antiviral nucleoside NHC or a prodrug or a salt thereof is administered twice daily, as individual doses of an amount of about 50 mg, about 100 mg, about 200 mg, about 300 mg, about 400 mg, about 500 mg, about 600 mg, about 700 mg, about 800 mg, about 1000 mg, about 1200 mg, about 1400 mg, or about 1600 mg. In aspects of such embodiments, the antiviral nucleoside NHC or a prodrug or a salt thereof is administered twice daily, as individual doses of an amount of about 200 mg. In aspects of such embodiments, the antiviral nucleoside NHC or a prodrug or a salt thereof is administered twice daily, as individual doses of an amount of about 300 mg. In aspects of such embodiments, the antiviral nucleoside NHC or a prodrug or a salt thereof is administered twice daily, as individual doses of an amount of about 400 mg. In aspects of such embodiments, the antiviral nucleoside is administered twice daily, as individual doses an amount of about 500 mg. In aspects of such embodiments, the antiviral nucleoside NHC or a prodrug or a salt thereof is administered twice daily, as individual doses of an amount of about 600 mg. In aspects of such embodiments, the antiviral nucleoside is administered twice daily, as individual doses of an amount of about 700 mg. In aspects of such embodiments, the antiviral nucleoside NHC or a prodrug or a salt thereof is administered twice daily, as individual doses of administered at an amount of about 800 mg.

In embodiments of the treatment methods disclosed herein, the antiviral nucleoside NHC or a prodrug or a salt thereof is administered in a dose of an amount of from about 1 mg/kg of animal body weight to about 500 mg/kg of animal body weight, or from about 10 mg/kg of animal body weight to about 100 mg/kg of animal body weight, or from about 1 mg/kg of animal body weight to about 10 mg/kg of animal body weight, or from about 10 mg/kg of animal body weight to about 50 mg/kg of animal body weight, or from about 20 mg/kg of animal body weight to about 40 mg/kg of animal body weight, or from about 50 mg/kg of animal body weight to about 100 mg/kg of animal body weight, or from about 100 mg/kg of animal body weight to about 500 mg/kg of animal body weight

The disclosure further relates to methods of providing antiviral prophylaxis, said method comprising administering to a subject in need thereof a therapy that comprises an antiviral nucleoside NHC or a prodrug or a salt thereof; wherein the antiviral nucleoside is administered once daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the antiviral nucleoside NHC or a prodrug or a salt thereof is administered once daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the antiviral nucleoside NHC or a prodrug or a salt thereof is administered once daily for 3 to 14 days, such as for 3 days, 4 days, 5 days, 6 days, 7 days, 8 days, 9 days, 10 days, 11 days, 12 days, 13 days, or 14 days.

The disclosure further relates to methods of providing antiviral prophylaxis, said method comprising administering to a subject in need thereof a therapy that comprises an antiviral nucleoside NHC or a prodrug or a salt thereof; wherein the antiviral nucleoside is administered twice daily for 1 to 42 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In specific embodiments, the antiviral nucleoside NHC or a prodrug or a salt thereof is administered twice daily for 1 to 21 days, such as for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In specific embodiments, the antiviral nucleoside NHC or a prodrug or a salt thereof is administered twice daily for 3 to 14 days, such as for 3 days, 4 days, 5 days, 6 days, 7 days, 8 days, 9 days, 10 days, 11 days, 12 days, 13 days, or 14 days.

In an embodiment, the antiviral nucleoside NHC or a prodrug or a salt thereof is administered orally. In an embodiment, the antiviral nucleoside NHC or a prodrug or a salt thereof is administered in the form of an oral pill or tablet. In an embodiment, the antiviral nucleoside NHC or a prodrug or a salt thereof is administered in the form of a liquid. In an embodiment, the antiviral nucleoside NHC or a prodrug or a salt thereof is administered in the animals' feed. In an embodiment, the antiviral nucleoside NHC or a prodrug or a salt thereof is administered in the animals' drinking water. In an embodiment, the antiviral nucleoside NHC or a prodrug or a salt thereof is administered topically. In an embodiment, the antiviral nucleoside NHC or a prodrug or a salt thereof is administered to buccal tissues. In an embodiment, the antiviral nucleoside NHC or a prodrug or a salt thereof is administered in the form of a nasal spray. In an embodiment, the antiviral nucleoside NHC or a prodrug or a salt thereof is administered by injection. In an embodiment, the antiviral nucleoside NHC or a prodrug or a salt thereof is administered in the form of an implant. In an embodiment, the antiviral nucleoside NHC or a prodrug or a salt thereof is administered to the eye of the animal. In an embodiment, the antiviral nucleoside NHC or a prodrug or a salt thereof is administered to the ear of the animal.

In an embodiment, the antiviral nucleoside NHC or a prodrug or a salt thereof is administered to all animals of a herd or collection of animals which contains some infected animals and some uninfected animals. In an embodiment, the antiviral nucleoside NHC or a prodrug or a salt thereof is administered to animals that have been exposed or potentially exposed to a virus but not yet developed symptoms of the viral disease.

An embodiment of the invention is a method of preventing or treating a viral disease in an animal comprising administering to the animal a pharmaceutical composition comprising an antiviral nucleoside β-D-N(4)-hydroxycytidine or a prodrug or salt thereof and a pharmaceutically acceptable carrier and wherein the viral disease is Porcine Reproductive and Respiratory Syndrome (PRRS), Bovine Respiratory Disease (BRD), Equine Influenza, Canine Distemper, Canine Respiratory Disease, Feline Infectious Peritonitis, Feline Viral Rhinotracheitis, or Feline Immunodeficiency.

In another embodiment, the administration is oral, topical or by injection.

In another embodiment, the animal is a swine and the disease is Porcine Reproductive and Respiratory Syndrome (PRRS). In another embodiment, the animal is a bovid and the disease is bovine Respiratory Disease (BRD). In another embodiment, the animal is a horse and the disease is Equine Influenza. In another embodiment, the animal is a dog and the disease is Canine Distemper or Canine Respiratory disease. In another embodiment, the animal is a cat and the disease is Feline Infectious Peritonitis, Feline Viral Rhinotracheitis, or Feline Immunodeficiency.

In another embodiment, the pharmaceutical composition is administered in a single dose or in multiple doses administered at multiple times.

An alternative embodiment is a method of preventing or treating a viral disease in an animal comprising administering to the animal a pharmaceutical composition comprising β-D-N(4)-hydroxycytidine or a prodrug or salt thereof and a pharmaceutically acceptable carrier and wherein the viral disease results from a SARS-CoV-2 infection in the animal and, wherein the animal is a swine, a bovid, a horse, a dog or a cat.

In another embodiment, the administration of the pharmaceutical composition to the animal does not interfere with the vaccination strategy of the animal.

An alternative embodiment is a pharmaceutical composition comprising an antiviral nucleoside β-D-N(4)-hydroxycytidine or a prodrug or salt thereof and a pharmaceutically acceptable carrier for inducing an antiviral response in an animal, wherein the animal is a swine, a bovid, a horse, a dog or a cat, the animal is suffering from or is susceptible to a viral disease and the viral disease is Porcine Reproductive and Respiratory Syndrome (PRRS), Bovine Respiratory Disease (BRD), Equine Influenza, Canine Distemper, Canine Respiratory Disease, Feline Infectious Peritonitis, Feline Viral Rhinotracheitis, or Feline Immunodeficiency.

In another embodiment, the animal is a swine and the disease is Porcine Reproductive and Respiratory Syndrome (PRRS). In another embodiment, the animal is a bovid and the disease is bovine Respiratory Disease (BRD). In another embodiment, the animal is a horse and the disease is Equine Influenza. In another embodiment, the animal is a dog and the disease is Canine Distemper or Canine Respiratory disease. In another embodiment, the animal is a cat and the disease is Feline Infectious Peritonitis, Feline Viral Rhinotracheitis, or Feline Immunodeficiency.

In another embodiment, the antiviral response is the amount of virus in the animal remains substantially the same.

In another embodiment, the antiviral response is the virus load of the animal decreases.

In another embodiment, the antiviral response is the virus load of the animal is substantially eliminated.

In another embodiment, the administration is oral.

In another embodiment, the oral administration is in the form of a tablet or a liquid.

In another embodiment, the oral administration is in the animal's drinking water.

In another embodiment, the oral administration is in the animal's feed.

In another embodiment, the administration is topical.

In another embodiment, the administration is by injection.

An alternative embodiment is a method of treating a viral infection, wherein the method comprises administering to the animal a pharmaceutical composition comprising an antiviral nucleoside β-D-N(4)-hydroxycytidine or a prodrug or salt thereof and a pharmaceutically acceptable carrier; wherein the viral infection is selected from the group consisting of Porcine Reproductive and Respiratory Syndrome virus (PRRS-1/PRRS-2), Bovine Leukemia virus (BLV), Bovine Corona Virus (BCV), Bovine Respiratory Syncytial Virus (BRSV), Bovine Rotavirus, Bovine Parainfluenza Virus 3, Bovine Viral Diarrhea Virus (BVDV), Equine Influenza Virus, Canine Distemper virus, Canine Influenza virus (CIV), Coronaviridae (FIPV), Feline Calicivirus, or Feline Immunodeficiency virus (FIV),

An alternative embodiment is a method of treating a viral infection, wherein the method comprises administering to the animal a pharmaceutical composition comprising an antiviral nucleoside β-D-N(4)-hydroxycytidine or a prodrug or salt thereof and a pharmaceutically acceptable carrier; wherein the viral infection is selected from the group consisting of Porcine Reproductive and Respiratory Syndrome virus (PRRS-1/PRRS-2), Bovine Leukemia virus (BLV), Bovine Corona Virus (BCV), Bovine Respiratory Syncytial Virus (BRSV), Bovine Rotavirus, Bovine Parainfluenza Virus 3, Bovine Viral Diarrhea Virus (BVDV), Equine Influenza Virus, Canine Distemper virus, Coronaviridae (FIPV), Feline Calicivirus, or Feline Immunodeficiency virus (FIV) In another embodiment, the viral infection is Porcine Reproductive and Respiratory Syndrome virus (PRRS-1/PRRS-2). In another embodiment, the viral infection is Bovine Leukemia virus (BLV). In another embodiment, the viral infection is Bovine Corona Virus (BCV). In another embodiment, the viral infection is Bovine Respiratory Syncytial Virus (BRSV). In another embodiment, the viral infection is Bovine Rotavirus. In another embodiment, the viral infection is Bovine Parainfluenza Virus 3. In another embodiment, the viral infection is Bovine Viral Diarrhea Virus (BVDV). In another embodiment, the viral infection is Equine Influenza Virus. In another embodiment, the viral infection is Canine Distemper virus. In another embodiment, the viral infection is Canine Influenza virus (CIV). In another embodiment, the viral infection is Coronaviridae (FIPV). In another embodiment, the viral infection is Feline Calicivirus. In another embodiment, the viral infection is Feline Immunodeficiency virus (FIV).

An alternative embodiment is a method of providing antiviral prophylaxis, wherein the method comprises administering to the animal a pharmaceutical composition comprising an antiviral nucleoside β-D-N(4)-hydroxycytidine or a prodrug or salt thereof and a pharmaceutically acceptable carrier; wherein the viral infection is selected from the group consisting of Porcine Reproductive and Respiratory Syndrome virus (PRRS-1/PRRS-2), Bovine Leukemia virus (BLV), Bovine Corona Virus (BCV), Bovine Respiratory Syncytial Virus (BRSV), Bovine Rotavirus, Bovine Parainfluenza Virus 3, Bovine Viral Diarrhea Virus (BVDV), Equine Influenza Virus, Canine Distemper virus, Canine Influenza virus (CIV), Coronaviridae (FIPV), Feline Calicivirus, or Feline Immunodeficiency virus (FIV).

Another embodiment is a method of providing antiviral prophylaxis, wherein the method comprises administering to the animal a pharmaceutical composition comprising an antiviral nucleoside β-D-N(4)-hydroxycytidine or a prodrug or salt thereof and a pharmaceutically acceptable carrier; wherein the viral infection is transmitted via the respiratory system or the upper respiratory tract. In another embodiment, the viral infection is an infection that is transmitted through the air. In yet another embodiment, the viral disease is transmitted via the gastrointestinal tract.

In another embodiment, the viral infection is Porcine Reproductive and Respiratory Syndrome virus (PRRS-1/PRRS-2). In another embodiment, the viral infection is Bovine Leukemia virus (BLV). In another embodiment, the viral infection is Bovine Corona Virus (BCV). In another embodiment, the viral infection is Bovine Respiratory Syncytial Virus (BRSV). In another embodiment, the viral infection is Bovine Rotavirus. In another embodiment, the viral infection is Bovine Parainfluenza Virus 3. In another embodiment, the viral infection is Bovine Viral Diarrhea Virus (BVDV). In another embodiment, the viral infection is Equine Influenza Virus. In another embodiment, the viral infection is Canine Distemper virus. In another embodiment, the viral infection is Canine Influenza virus (CIV). In another embodiment, the viral infection is Coronaviridae (FIPV). In another embodiment, the viral infection is Feline Calicivirus. In another embodiment, the viral infection is Feline Immunodeficiency virus (FIV).

An embodiment of the invention is a method of preventing or treating a viral disease in an animal comprising administering to the animal a pharmaceutical composition comprising an antiviral nucleoside β-D-N(4)-hydroxycytidine or a prodrug or salt thereof and a pharmaceutically acceptable carrier and wherein the viral disease results from a RNA virus infection in the animal.

In another embodiment, the animal is a swine, a bovid, a horse, a dog, or a cat.

In another embodiment, the animal is a swine and the disease is Porcine Reproductive and Respiratory Syndrome (PRRS).

In another embodiment, the animal is a bovid and the disease is bovine Respiratory Disease (BRD).

In another embodiment, the animal is a horse and the disease is Equine Influenza.

In another embodiment, the animal is a dog and the disease is Canine Distemper or Canine Respiratory disease.

In another embodiment, the animal is a cat and the disease is Feline Infectious Peritonitis, Feline Viral Rhinotracheitis, or Feline Immunodeficiency.

In another embodiment, the RNA virus is a single strand RNA (ssRNA) virus.

In another embodiment, the RNA virus is a double strand RNA (dsRNA) virus.

In another embodiment, the RNA virus is a positive stand RNA virus.

In another embodiment, the positive stand RNA virus is Arteriviridae, Astroviridae, Caliciviridae, Coronaviridae, Flaviviridae, Hepeviridae, Nodaviridae, Picornaviridae, Toroviridae or Togaviridae.

In another embodiment, the RNA virus is a negative strand RNA virus.

In another embodiment, the RNA virus is an ambisense RNA virus.

In another embodiment, the negative strand RNA virus is Arenaviridae, Bornaviridae, Bunyaviridae, Filoviridae, Nymaviridae, Orthmyxoviridae, Paramyxoviridae, Pneumovirida, or Rhabdoviridaeb.

An embodiment of the invention is method of preventing or treating a viral disease in an animal comprising administering to the animal a pharmaceutical composition comprising an antiviral nucleoside β-D-N(4)-hydroxycytidine or a prodrug or salt thereof and a pharmaceutically acceptable carrier and wherein the viral disease is caused by Porcine Influenza virus, Porcine Rotavirus, Feline Infectious Peritonitis virus (FIPV) or Feline Calicivirus.

In another embodiment, the administration is by oral, topical or injectable route, preferably oral.

In a preferred embodiment, the animal is a swine and the disease is caused by Porcine influenza virus or Porcine Rotavirus.

In a preferred embodiment, the animal is a swine and the disease is caused by Porcine influenza virus.

In a preferred embodiment, the animal is a swine and the disease is caused by Porcine Rotavirus.

In a preferred embodiment, the animal is a cat and the disease is caused by Feline Infectious Peritonitis virus or Feline Calicivirus.

In a preferred embodiment, the animal is a cat and the disease is caused by Feline Infectious Peritonitis virus.

In a preferred embodiment, the animal is a cat and the disease is caused by Feline Calicivirus.

In another embodiment, the pharmaceutical composition is administered in a single dose or in multiple doses administered at multiple times.

Another embodiment of the invention is a method of preventing or treating a viral disease in an animal comprising administering to the animal a pharmaceutical composition comprising β-D-N(4)-hydroxycytidine or a prodrug or salt thereof and a pharmaceutically acceptable carrier and wherein the viral disease results from a SARS-CoV-2 infection in the animal and wherein the animal is a swine, a bovid, a horse, a dog or a cat.

Another embodiment of the invention is a pharmaceutical composition comprising an antiviral nucleoside β-D-N(4)-hydroxycytidine or a prodrug or salt thereof and a pharmaceutically acceptable carrier for use in preventing or treating a viral disease in an animal, wherein the animal is a swine, or a cat, the animal is suffering from or is susceptible to a viral disease is caused by Porcine Influenza virus, Porcine Rotavirus, viruses Feline Infectious Peritonitis virus, or Feline Calicivirus.

Another embodiment of the invention is a pharmaceutical composition comprising an antiviral nucleoside β-D-N(4)-hydroxycytidine or a prodrug or salt thereof and a pharmaceutically acceptable carrier for inducing an antiviral response in an animal, wherein the animal is a swine, or a cat, the animal is suffering from or is susceptible to a viral disease and the viral disease is caused by Porcine Influenza virus, Porcine Rotavirus, Feline Infectious Peritonitis virus or Feline Calicivirus.

In another embodiment, the antiviral response is the amount of virus in the animal remains substantially the same.

In another embodiment, the antiviral response is the amount of virus in the animal decreases.

In another embodiment, the antiviral response is the amount of virus in the animal is substantially eliminated.

Another embodiment of the invention is a method of treating a viral infection, wherein the method comprises administering to an animal a pharmaceutical composition comprising an antiviral nucleoside β-D-N(4)-hydroxycytidine or a prodrug or salt thereof and a pharmaceutically acceptable carrier; wherein the viral infection is selected from the group consisting of Porcine Rotavirus, Porcine Influenza virus, Feline Infectious Peritonitis virus (FIPV), or Feline Calicivirus.

Another embodiment of the invention is a method of providing antiviral prophylaxis, wherein the method comprises administering to an animal a pharmaceutical composition comprising an antiviral nucleoside β-D-N(4)-hydroxycytidine or a prodrug or salt thereof and a pharmaceutically acceptable carrier; wherein the viral infection is selected from the group consisting of Porcine Rotavirus, Porcine Influenza virus, Feline Infectious Peritonitis virus (FIPV), or Feline Calicivirus.

Another embodiment of the invention is a method of preventing or treating a viral disease in an animal comprising administering to the animal a pharmaceutical composition comprising an antiviral nucleoside β-D-N(4)-hydroxycytidine or a prodrug or salt thereof and a pharmaceutically acceptable carrier and wherein the viral disease is caused by Coronaviridae virus.

In another embodiment, the animal is a feline or a porcine.

Porcine means having to do with pigs or swine.

Feline means having to do with cat or other members of the cat family.

Additional embodiments of the disclosure include the pharmaceutical compositions, combinations, uses and methods set forth in above, wherein it is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

### EXAMPLES

### Example 1 CPE Antiviral Assay for Feline Infectious Peritonitis Virus (FIPV) in Crandell-Rees Feline Kidney (CrFK) Cells

Crandell-Rees Feline Kidney (CRFK, ATCC CCL-94) cells were cultivated in DMEM medium containing 2% FBS, GlutaMax, D-glucose, Na-pyruvate, non-essential amino acids and pencillin/streptomycin. Cells were harvested for viral infection by trypsinization. 50 ml CRFK cells (1.5×10⁵ cells/ml) were infected Feline Infectious Peritonitis (FIPV) strain 79-1146. The conditions of infection were optimized for an CPE assay window at four days post infection. FIPV-infected CRFK cells were mixed with NHC compound at serial ⅓ dilutions from 10 to 0.000508 μM final concentration. Subsequently each dilution was plated in replicates at 100 μl/well into 96-well plates (Costar 3903 or 3904). Cells were incubated in a humidified incubator at 37° C. with 5% C02 for 96 hrs. The cytopathic effect (CPE) was determined using the CellTiterGlo Luminescent Cell Viability Assay (Promega) in a Tecan Infinite M1000 PRO plate reader according to the manufacturer's protocol. The effective concentration of NHC that reduced FIVP replication by 50% (EC₅₀) was calculated in a 4-parameter nonlinear regression model using GraphPad Prism 8.

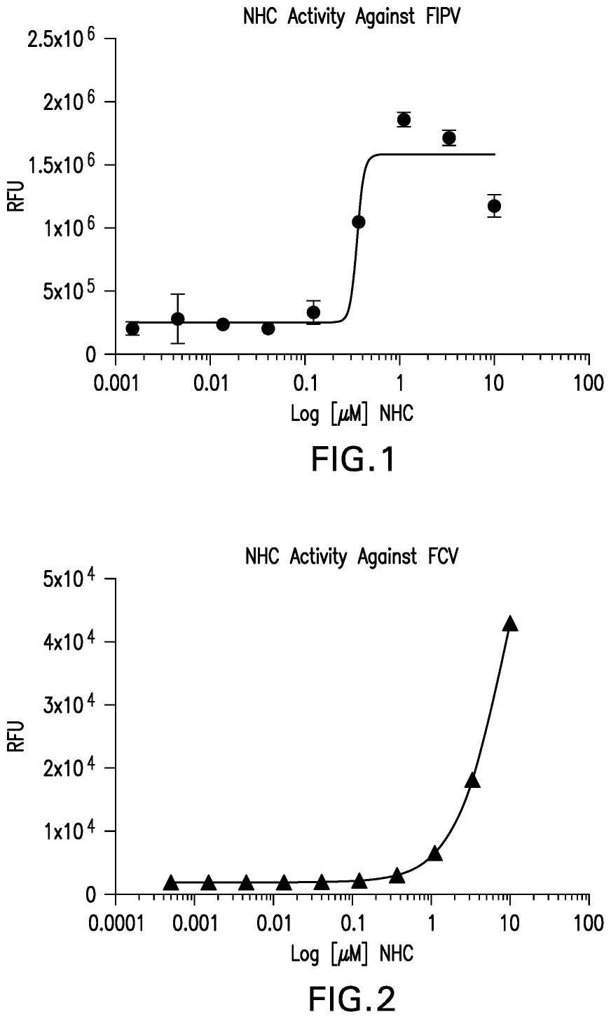

The results of this test show the concentration of NHC that effectively inhibits the FIP virus in CRFK cells. See FIG. 1 and Table 1 below.

TABLE 1

| Compound | EC 50 (μM) |
|---|---|
| GC-376 | 1.816-3.0 |
| MK-0608 | >30 |
| Remdesivir | 0.7352-0.97 |
| NHC | 0.3336-0.37 |

MK-0608 is a potent and orally bioavailable inhibitor of HCV replication in vitro (Carroll et al, ANTIMICROBIAL AGENTS AND CHEMOTHERAPY, March 2009, p. 926-934).

GC-376 is a a 3C-like protease inhibitor known for treating various forms of acquired feline infectious peritonitis (Pedersen et al. J. of Feline Med Surg. 8 2(4):378-392).

Remdesivir is a prodrug of an adenosine triphosphate (ATP) analog, with potential antiviral activity against a variety of RNA viruses. APA National Center for Biotechnology Information (2021). PubChem Compound Summary for CID 121304016, Remdesivir. Retrieved Jul. 21, 2021 from https://pubchem.ncbi.nlm.nih.gov/compound/Remdesivir.

NHC is β-D-N(4)-hydroxycytidine and is described above.

### Example 2 CrFK Cell Based Antiviral Activity of NHC Against Feline Calicivirus (FCV)

Monolayers of Crandell-Rees Feline Kidney (CrFK) cells grown in 96-well plates (Greiner Cat #655090) in 100 μl/well M6B8 medium supplemented with 5% FCS and 0.15% NaHCO₃ at a density of 2×10⁵ cells/ml were infected with feline calicivirus (FCV) field strain 255 at multiplicities of infection of 0.01 and 0.001. NHC was added in ⅓ serial dilutions to the infected cells to yield a test concentration range of from 10 μM to 0.000508 μM and cultures were incubated for up to 48 hours at 37° C. and 5% C02. The extent of viral replication was assessed by determining the degree of FCV-induced cytopathic effect (CPE) using the CellTiterGlo® Luminescent Cell Viability Assay (Promega Cat #G7571) by adding the reagent 1:1 (v/v) to the culture plates that have been equilibrated to room temperature for 30 minutes. Plates were incubated for 10 minutes at room temperature and subsequently read for luminescence in a Biotek Cytation5. The effective concentration of NHC that reduced FCV replication by 50% (EC₅₀) was calculated in a 4-parameter nonlinear regression model using GraphPad Prism 8. The ED₅₀ of NHC against Feline calicivirus is 3.8 μM (FIG. 2).

### Example 3 Anti-Porcine Rotavirus Activity of β-D-N(4)-Hydroxycytidine (NHC)

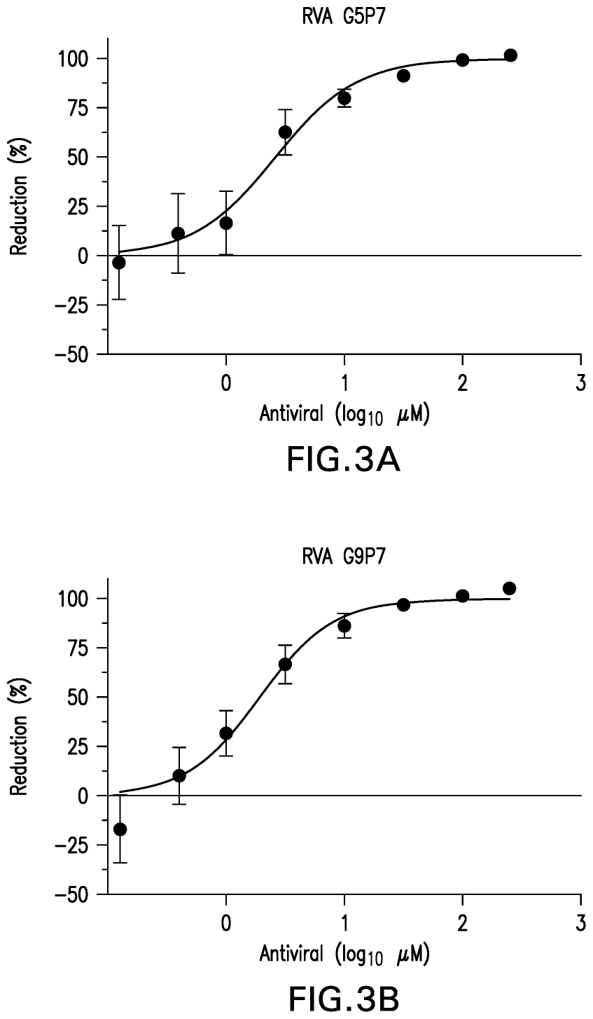

The anti-rotaviral activity of NHC against swine rotaviruses was tested in a cell culture-based assay. MA-104 (Rhesus monkey kidney tissue) cells were cultured and harvested according to standard procedures. The harvested MA-104 cell suspension was seeded onto 96-well plates (Greiner CELLSTAR® black, Sigma-Aldrich® M0562) in 200 μl/well M6B8 medium with 5% FBS at a density of 3×10⁴ cells/mL. After 4 days of culturing, the monolayers were washed and the cells were infected with rotavirus A by adding 100 μl/well of 3630 TCID₅₀/mL Porcine rotavirus A strain of serotype G5P7 (ATCC, VR-892) or strain G9P7 (MAH Worthington, USA). After 1 hour of infection, NHC was added in ⅓ serial dilutions to yield a test concentration range from 270 μM to 123 nM and cells were cultured for 3 days at 37° C. under 5% C02. Replication inhibition of the Rotavirus A strains by NHC was quantified using a mouse-anti-Rotavirus A strain-specific monoclonal antibody (Ingenasa, #1JF10) followed by FITC-fluorochrome conjugated goat-anti-mouse antibody (Nordic) and DAPI (Sigma Aldrich) nuclear staining in an immunofluorescent assay. The extent of immunofluorescence was quantified as relative fluorescence units (RFU) at 485 nm excitation and 528 nm emission using a Cytation 5 cell imaging multimode reader and Gen5 software version 3.10 (Agilent BioTek). The inhibitory concentration of NHC that reduced the rotavirus replication by 50% (IC₅₀) was calculated by a 4-parameter nonlinear regression model using GraphPad Prism 8. The results demonstrate that NHC is able to inhibit the replication of porcine rotavirus strains G5P7 and G9P7. The effective concentration of NHC where the replication of porcine rotavirus is reduced by half (EC₅₀) is 2.6 μM for the G5P7 strain (FIG. 3a). and 1.9 μM for the G9P7 strain (FIG. 3b).

### Example 4 Anti-Swine Influenza Virus a Activity of β-D-N(4)-Hydroxycytidine (NHC)

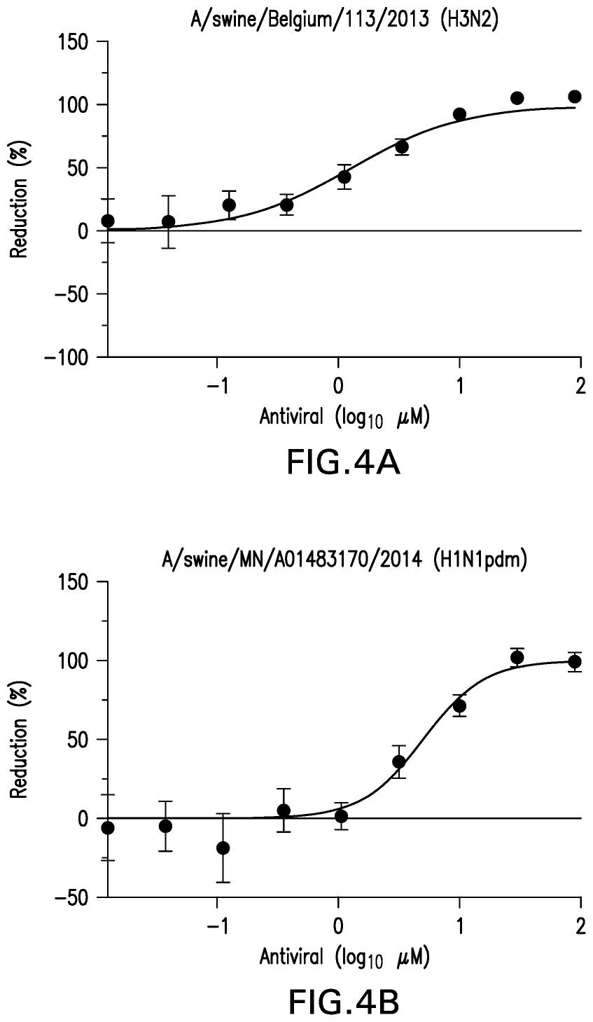

The antiviral activity of NHC against swine influenza viruses was tested in a cell culture-based assay. MDCK (Madin-Darby Canine Kidney) cell suspension was seeded onto 96-well plates (Greiner CELLSTAR® black, Sigma-Aldrich® M0562) in 200 μl/well DMEM medium with 5% FBS at a density of 6×10⁴ cells/mL. After 3 days of culturing, the monolayers were washed, and the cells were infected with swine influenza virus (SIV) by adding 100 μl/well of 5000 TCID₅₀/mL strain A/swine/MN/A01483170/2014 (H1N1 pdm) or 2500 TCID₅₀/mL of strain A/swine/Belgium/113/2013 (H3N2). After 1 hour of infection, NHC was added in ⅓ serial dilutions to yield a test concentration range from 270 μM to 123 nM and cells were cultured for 1 day at 37° C. under 5% C02. Replication inhibition of the SIV strains by NHC was quantified using a mouse-anti-SIV virus specific monoclonal antibody (MSD AH, #HB-65) followed by Alexa Fluor 488-fluorochrome conjugated goat-anti-mouse antibody (Invitrogen) and DAPI (Sigma Aldrich) nuclear staining in an immunofluorescent assay. The extent of immunofluorescence was imaged and quantified as relative fluorescence units (RFU) at 488 nm excitation and 525 nm emission using a Cytation 5 cell imaging multimode reader and Gen5 software version 3.10 (Agilent BioTek). The effective concentration of NHC that reduced SIV replication by 50% (EC₅₀) was calculated in a 4-parameter nonlinear regression model using GraphPad Prism 8. The effective concentration of NHC where the replication of swine influenza virus is reduced by half (EC₅₀) is 1.3 μM for the H3N2 strain (FIG. 4a). and 5.2 μM for the H1N1pdm strain (FIG. 4b).

1. A method of preventing or treating a viral disease in an animal comprising administering to the animal the pharmaceutical composition of claim 7, wherein the viral disease is caused by Porcine Influenza virus, Porcine Rotavirus, Feline Infectious Peritonitis virus (FIPV) or Feline Calicivirus.

2. The method of claim 1, wherein the administration is by oral, topical or injectable route, preferably oral.

3. The method of claim 1, wherein the animal is a swine and the disease is caused by Porcine influenza virus or Porcine Rotavirus.

4. The method of claim 1, wherein the animal is a cat and the disease is caused by Feline Infectious Peritonitis virus or Feline Calicivirus.

5. The method of claim 1, wherein the pharmaceutical composition is administered in a single dose or in multiple doses administered at multiple times.

6. A method of preventing or treating a viral disease in an animal comprising administering to the animal the pharmaceutical composition of claim 7, wherein the viral disease results from a SARS-CoV-2 infection in the animal and wherein the animal is a swine, a bovid, a horse, a dog or a cat.

7. A pharmaceutical composition comprising an antiviral nucleoside β-D-N(4)-hydroxycytidine or a prodrug or salt thereof and a pharmaceutically acceptable carrier.

8. A method of inducing an antiviral response in an animal comprising administering to the animal the pharmaceutical composition of claim 7, wherein the animal is a swine, or a cat, the animal is suffering from or is susceptible to a viral disease and the viral disease is caused by Porcine Influenza virus, Porcine Rotavirus, Feline Infectious Peritonitis virus or Feline Calicivirus.

9. The method of claim 8, wherein the antiviral response is the amount of virus in the animal remains substantially the same.

10. The method of claim 8, wherein the antiviral response is the amount of virus in the animal decreases.

11. The method of claim 8, wherein the antiviral response is the amount of virus in the animal is substantially eliminated.

12. A method of treating a viral infection, wherein the method comprises administering to an animal the pharmaceutical composition of claim 7, wherein the viral infection is selected from the group consisting of Porcine Rotavirus, Porcine Influenza virus, Feline Infectious Peritonitis virus (FIPV), or Feline Calicivirus.

13. A method of providing antiviral prophylaxis, wherein the method comprises administering to an animal the pharmaceutical composition of claim 7, wherein the viral infection is selected from the group consisting of Porcine Rotavirus, Porcine Influenza virus, Feline Infectious Peritonitis virus (FIPV), or Feline Calicivirus.

14. A method of preventing or treating a viral disease in an animal comprising administering to the animal the pharmaceutical composition of claim 7, wherein the viral disease is caused by Coronaviridae virus.

15. The method of claim 14, wherein the animal is a feline or a porcine.
